(19) United States
(12) Patent Application Publication
(10) Pub. No.: US 2014/0206841 A1
(43) Pub. Date: Jul. 24, 2014

(54) Peptide Synthesis Apparatus and Methods Using Infrared Energy

(71) Applicant: PROTEIN TECHNOLOGIES, INC., Tucson, AZ (US)

(72) Inventors: Mahendra Menakuru, Tucson, AZ (US); Alexander Waddell, Tucson, AZ (US)

(73) Assignee: Protein Technologies, Inc., Tucson, AZ (US)

(21) Appl. No.: 13/745,631

(22) Filed: Jan. 18, 2013

(51) Int. Cl. C07K 1/04

(52) U.S. Cl. CPC C07K 1/04
USPC 530/334; 422/131; 422/119

(57) ABSTRACT

Apparatus and methods utilizing infrared energy for heating reactions associated with peptide synthesis, such as activation, deprotection, coupling, and cleavage. Thorough agitation of the contents of reaction vessels during heating and real-time monitoring and adjustment of temperature and/or reaction duration are also described. Existing peptide synthesizers may be retrofitted to include an infrared energy source.

### BACKGROUND OF THE INVENTION

Proteins are chains of amino acids whose sequences are specified by gene sequences. Relatively short chains of amino acids are called peptides. Peptides and proteins have several roles in the body including structure (i.e. collagen in skin), catalysis (i.e. enzymes), regulation and protection (i.e. hormones and antibodies), and substance transport (i.e. oxygen transport by hemoglobin). Peptides are used in various applications, including basic biological research of protein structure and function, biological mechanisms and disease pathology, as well as peptide-based drugs and peptide-based materials including scaffolds for tissue engineering.

Amino acids are composed of an amino group on one end and a carboxylic acid group on the other end as well as a side-chain, or R-group. The amino and carboxylic acid groups can react and form an amide or peptide bond. R group properties cause amino acid chains to fold into specific shapes. Each sequence produces a differently shaped protein or peptide, capable of performing its unique function.

Cells are equipped with machinery that synthesizes proteins and peptides from gene sequences. In 1963, Bruce Merrifield developed a method for synthesizing peptides on a solid resin support. Known as solid-phase peptide synthesis (SPPS), this method allows peptides to be synthesized in any sequence, with any amino acid(s) on an automated synthesizer.

Basically, SPPS is the stepwise addition of free amino acids to a growing polypeptide chain attached to a solid resin support via a linker molecule. Protecting groups on the free amino groups prevent them from reacting, while side-chain protecting groups prevent reactive side chain groups from reacting, and forming unwanted byproducts and impurities. Chain elongation begins with the removal of the protecting group from the last amino acid attached to the polypeptide chain. This step is known as “deprotection.” Following deprotection, the carboxylic acid group of the next free amino acid to be added is activated to make it more reactive. The activated free amino acid can then form an amide, or peptide bond with the free amino group of the growing peptide chain. This step is called “coupling.” Deprotection and coupling are repeated until the desired chain length is achieved. Finally, the polypeptide chain is removed from the resin support in a process known as “cleavage.”

In general, there are two types of protecting groups used in solid-phase peptide synthesis: Boc (t-Butoxycarbonyl) and Fmoc (9-fluorenylmethyloxycarbonyl). Different chemistries are performed depending on which protecting group is used. The original chemistry for peptide synthesis was based on the Boc protecting group and the Benzyl (Bzl) side-chain protecting group. Removal of the Boc group during deprotection is accomplished using trifluoroacetic acid (TFA). Cleavage and removal of the Bzl side-chain protecting groups is accomplished using hydrofluoric acid (HF) which is an even stronger acid. HF must be handled very carefully because it can dissolve bone. A milder chemistry was developed in the 1980's based on the Fmoc protecting group and the tent-Butyl (t-Bu) side-chain protecting group. Removal of the Fmoc group during deprotection is accomplished using base, typically a solution of 20-25% piperidine. Cleavage and removal of the t-Bu side-chain protecting groups is accomplished using trifluoroacetic acid (TFA).

The highly repetitive nature of SPPS makes it well suited for automation. One of the challenges of SPPS is the synthesis of long or difficult peptides. Growing peptide chains can undergo intramolecular or intermolecular interactions with themselves or neighboring peptide chains in a phenomenon known as aggregation. Long sequences, or sequences containing high numbers of hydrophobic residues are particularly susceptible to aggregation, which along with sterically hindered side chains can obscure the reactive site of the growing peptide chain, resulting in synthetic peptides with lower purities and yields.

Several methods which have been shown to overcome these difficulties include the use of pseudoproline dipeptides, more efficient activators, such as HATU or HCTU, the choice of solvent and low-loaded resins. In each case, the synthetic challenge has been overcome by the use of one or more of these tools.

The application of heat, typically using oil baths, heating elements, or microwaves, has emerged as an additional tool for overcoming these challenges. Microwave heating in particular has grown in popularity in recent years, due to the speed with which small volumes can be raised to elevated temperatures.

While microwaves offer rapid heating, there are also a number of considerable disadvantages associated with this technology. All commercial single-mode microwave reactors currently available allow irradiation of only a single vessel. Thus it is not possible to perform microwave synthesis of multiple peptides in parallel. Furthermore, limitations in the reaction vessel and mixing options available on microwave synthesizers make scale-up of microwave conditions practically impossible.

A system which could match the advantages of microwave heating (rapid, efficient) while eliminating the disadvantages (e.g., serial synthesis, limited scale) would be a powerful new tool at the disposal of the synthetic peptide chemist.

### SUMMARY OF THE INVENTION

This disclosure relates to a novel peptide synthesizer with infrared (IR) heating. While heating with IR is as rapid as that with microwaves, parallel synthesis is possible because multiple reaction vessels can be heated with IR simultaneously. Different types of mixing also are possible, such as vortex mixing (or vortex mixing with nitrogen bubbling), which ensures that a homogeneous temperature distribution is maintained, making the synthesis operations scalable.

In one aspect, this disclosure relates to a method for the solid-phase synthesis of peptides comprising the application of IR energy to one or more of (a) deprotection of chemical protecting groups on an amino acid linked to a solid phase resin, (b) activation of an amino acid in preparation for coupling to the deprotected amino acid on resin, (c) coupling of the amino acids to form a peptide, (d) deprotecting, activating and coupling a third and succeeding amino acids into a peptide on the solid phase resin.

In another aspect, this disclosure relates to an apparatus for the accelerated synthesis of peptides by the solid phase method. In this aspect the invention comprises one or more reaction vessels and one or more infrared sources configured to heat the vessels, along with the other components of a synthesizer, e.g., a solid phase peptide support resin in the reaction vessel(s); frit(s) for maintaining the solid phase support resin in the vessel(s); and a means of moving fluid to and from the reaction vessel(s).

Various other purposes and advantages will become clear from its description in the specification that follows. Therefore, embodiments described herein include the features hereinafter fully described in the detailed description of the preferred embodiments, and particularly pointed out in the claims. However, such description discloses only some of the embodiments and the various ways in which the described embodiments may be practiced.

### BRIEF DESCRIPTION OF THE DRAWINGS

FIG. 1 is a perspective view of an embodiment of a peptide synthesis system utilizing IR energy.

FIG. 2A is a magnified cut-away view of the peptide synthesis system shown in FIG. 2B.

FIG. 2B shows the same embodiment as FIG. 1 but with the cover assembly down and partially cut away to reveal a reaction vessel and associated components.

FIG. 3 is a flow diagram showing the basic heating process logic for a system of the invention.

### DETAILED DESCRIPTION

Embodiments described herein relate to methods and apparatuses for the solid phase synthesis of peptides using infrared energy.

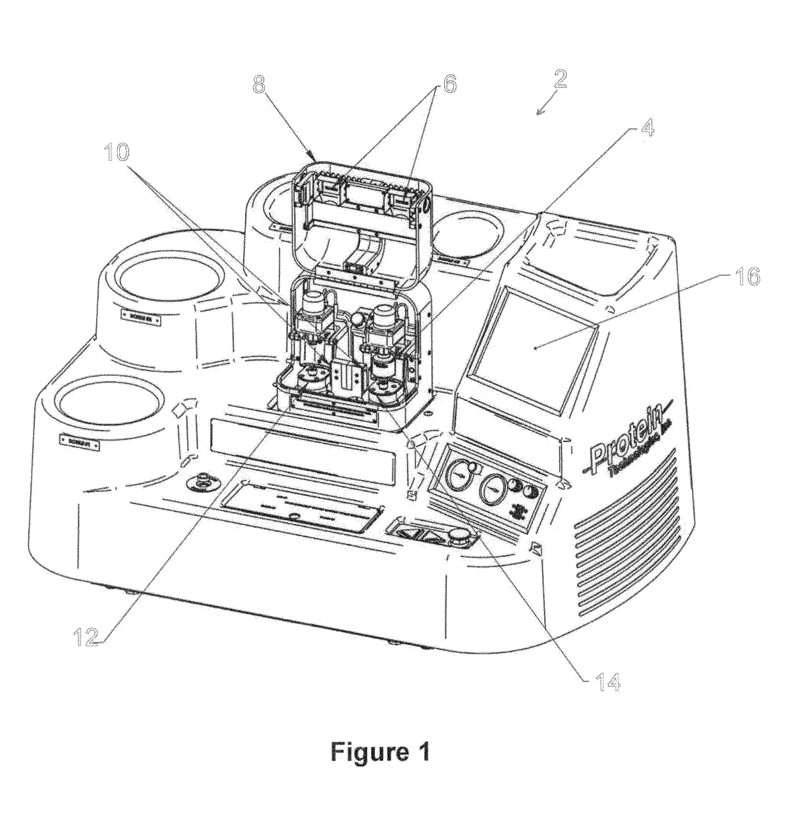

Turning to FIG. 1, a perspective view of a synthesizer embodiment 2 is shown. Reaction vessel 4 is configured to receive heating from one of infrared heating source 6 disposed upon hinged cover assembly 8. Infrared temperature sensors 10 provide feedback to the heating source 6 such that a heating cycle is initiated or stopped depending on whether a predetermined set point is reached.

Reaction vessel shaker assemblies 12 and 14 help provide consistent heating inside each reaction vessel 4. The synthesizer 2 includes various components (not shown) that enable it to operate and be controlled through, for example, actuating and control means 16 having a touch screen interface for a processor, software, etc. The IR monitoring, feedback and control portions of the synthesis instrument are known and can essentially be comprised of those set forth in US 20120080608 A1. The fluid delivery and membrane valve systems of the synthesis instruments may be essentially the same as those set forth in U.S. Pat. No. 5,203,368.

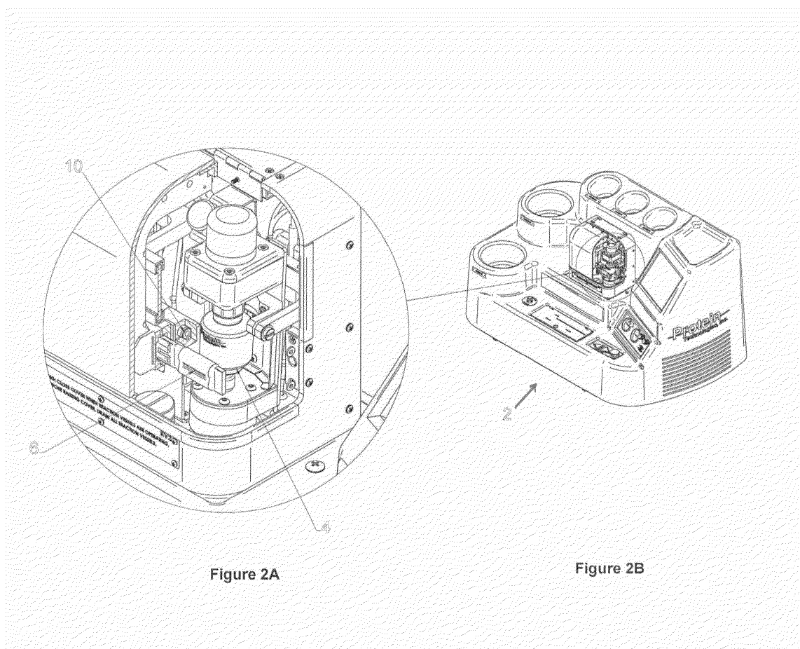

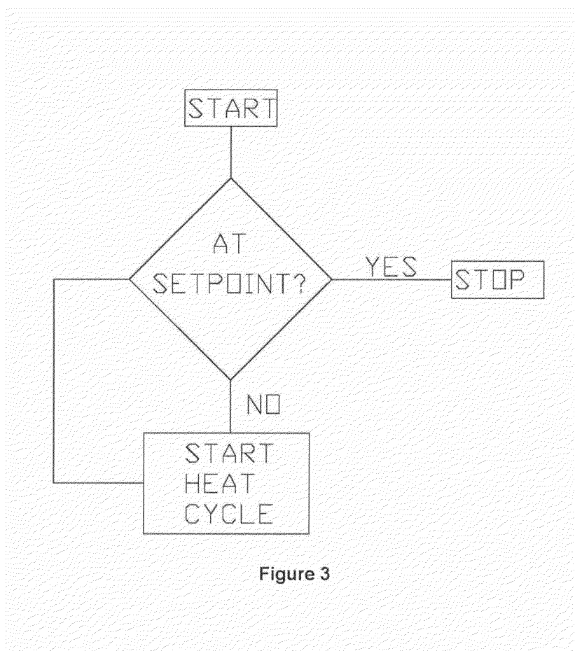

FIGS. 2A and 2B shows the embodiment of FIG. 1, but with the hinged cover assembly 8 closed (and partially cut away in 2A) such that IR heating sources 6 are in near proximity to each reaction vessel 4. FIG. 3 emphasizes the simplicity of design for effectively heating the reactants during synthesis, in the form of a flow diagram showing a heating process logic.

In a preferred embodiment the infrared source is custom-made and operates at 450 W. The apparatuses include an IR sensor for monitoring temperature. In a preferred embodiment the IR sensor provides real-time dynamic monitoring of the temperature, which is used to modulate the output of the infrared source. In a preferred embodiment the reaction vessels are made from borosilicate KG-133 glass.

In a preferred embodiment, a method comprises first treating a resin with an attached chemical protecting group or chemically-protected amino acid with a deprotection solution with concurrent application of infrared energy. The step may be repeated. The deprotection solution is removed and the resin is washed with an appropriate solvent. The first or next amino acid to be added to the resin may be added to the reaction vessel containing the resin, followed by activator solution and optionally a separate base.

Alternatively, the amino acid may be activated separately, for instance by the addition of base to a vial containing amino acid and activator, by the addition of activator and base solution to a vial containing amino acid, or by the addition of coupling additive and activator solution to a vial containing amino acid, before addition to the reaction vessel. Infrared energy is applied during the coupling step, which may occur with in situ activation or subsequent to activation.

After the coupling step, the solution is drained and the resin washed with an appropriate solvent. The coupling step may be repeated. The overall cycle of deprotection, washing, coupling, and washing may be repeated multiple times. When peptide synthesis is complete, an additional deprotection step may be necessary to remove the final protecting group prior to peptide cleavage. The resin may optionally be washed by an additional solvent and optionally dried prior to cleavage. To cleave the remaining side-chain protecting groups and cleave the peptide from the resin, a cleavage solution is added to the reaction vessel(s). Optionally infrared energy may be applied during the cleavage step, which may decrease the time necessary for this step. Upon completion, each mixture of cleaved peptide in cleavage solution is transferred to a collection vial.

In another preferred embodiment, a method comprises first treating a resin with an attached chemical protecting group or chemically-protected amino acid with a deprotection solution with concurrent application of infrared energy while monitoring the reaction solution in real time using UV detection. The step may be repeated, and the time or number of repetitions of the step may be determined by the UV measurements. The deprotection solution is removed and the resin is washed with an appropriate solvent.

The first or next amino acid to be added to the resin may be added to the reaction vessel containing the resin, followed by activator solution and optionally a separate base. Alternatively, the amino acid may be activated separately, for instance by the addition of base to a vial containing amino acid and activator, by the addition of activator and base solution to a vial containing amino acid, or by the addition of coupling additive and activator solution to a vial containing amino acid, before addition to the reaction vessel. Infrared energy is applied during the coupling step, which may occur with in situ activation or subsequent to activation. Optionally the length of the coupling step may be determined by the UV measurements made in real time during the deprotection step.

After the coupling step, the solution is drained and the resin washed with an appropriate solvent. The coupling step may be repeated. The overall cycle of deprotection, washing, coupling, and washing may be repeated multiple times. When peptide synthesis is complete, an additional deprotection step may be necessary to remove the final protecting group prior to peptide cleavage. The resin may optionally be washed by an additional solvent and optionally dried prior to cleavage. To cleave the remaining side-chain protecting groups and cleave the peptide from the resin, a cleavage solution is added to the reaction vessel(s). Optionally infrared energy may be applied during the cleavage step, which may decrease the time necessary for this step. Upon completion, each mixture of cleaved peptide in cleavage solution is transferred to a collection vial.

The methods may include agitation of the reaction mixture by means of bubbling using inert gas, vortex mixing, recirculation, or a combination of these.

In a preferred embodiment a method includes agitation by vortex mixing or recirculation for homogeneous heat distribution.

The activation step may occur separately or using an in situ activation method, with activator including but not limited to those of the carbodiimide, phosphonium, and aminium/uronium type, including but not limited to DCC, EDC, DIC, BOP, PyBOP, PyClOCK, HBTU, TBTU, HCTU, HATU, and COMU. Optionally an additive including but not limited to HOBt or Oxyma may be present.

As noted, the fluid delivery and membrane valve systems of the synthesis instruments may be essentially the same as those set forth in U.S. Pat. No. 5,203,368. However, reference to this application is not meant to imply limitation to any specific number of reaction vessels, amino acid bottles, or reagent bottles.

The amino acid source containers of this apparatus may contain the “common” amino acids for synthesizing proteins that are well known to those skilled in this art. These commercially available amino acids can be purchased in chemically protected form. Optionally these containers may contain other natural or unnatural amino acids or monomers.

### Non-Limiting Examples

ACP (65-74):
Val-Gln-Ala-Ala-Ile-Asp-Tyr-Ile-Asn-Gly-OH
Aib Enkephalin:
Tyr-Aib-Aib-Gly-Leu-NH₂

Synthesis: ACP (65-74) was synthesized on the Tribute® UV-IR peptide synthesizer at 100 μmol scale using Fmoc-Gly-Wang resin (loading 0.36 mmol/g). Deprotection was performed with 20% piperidine in DMF with 0.1 M HOBt for 30 seconds and 3 min at 50° C. Washes: DMF 6×30 sec. Coupling was performed using 0.5 mmol AA (5 eq.), 0.5 mmol (5 eq.) HBTU, and 1.0 mmol of DIEA (10 eq.) in 2.0 mL of DMF for 5 min at 50° C. Cleavage was performed using 95/2.5/2.5 TFA/TIS/water for 2 hours at room temperature. After precipitation in ice-cold ether, the resulting crude peptide was dried overnight.
Tyr-Aib-Aib-Phe-Leu-NH₂ was synthesized on the Tribute® peptide synthesizer at 50 μmol scale using Rink Amide ChemMatrix resin (loading 0.54 mmol/g). Deprotection was performed with 20% piperidine in DMF for 2×3 min at 77° C. Washes: DMF 6×30 sec. Coupling was performed using 0.5 mmol AA (10 eq.), 0.5 mmol COMU (10 eq.), and 1.0 mmol of DIEA (20 eq.) in 2.0 mL of DMF for 6 min at 77° C., except for Fmoc-Aib-OH, which was coupled in 6× excess. Cleavage was performed using 95/2.5/2.5 TFA/TIS/water for 2 hours. The mixture was precipitated and washed three times using 1:1 ether:hexanes, with centrifugation and decanting. The resulting crude peptides were dissolved in water and analyzed by HPLC and LC/MS.
Analysis: The crude peptides were analyzed on a Varian ProStar HPLC using a C18, 180 Å, 5 μm, 250×4.6 mm column (Agilent Polaris). ACP was analyzed over 50 minutes with a flow rate of 1 mL/min, and using a gradient of 5-95% B, where Buffer A is 0.1% TFA in water, and Buffer B is 0.1% TFA in acetonitrile, while Tyr-Aib-Aib-Phe-Leu-NH₂ was analyzed over 24 minutes using a gradient of 20-50% B. Detection was at 214 nm. Mass analysis was performed on a Shimadzu LCMS-2020 Single-Quad mass spectrometer, equipped with a C18, 100 Å, 2.6 μm, 50×2.1 mm column (Phenomenex Kinetex), over 7 minutes with a flow rate of 1 mL/min and using a gradient of 5-50% B where Buffer A is 0.1% formic acid in water and Buffer B is 0.1% formic acid in acetonitrile.
Results: ACP (65-74) was synthesized using infrared heating on the Tribute® UV-IR with a crude purity of 92%. LC-MS analysis confirmed that the correct peptide was synthesized. Tyr-Aib-Aib-Phe-Leu-NH₂ was synthesized using infrared heating on the Tribute® UV-IR with a crude purity of 89%. LC-MS analysis confirmed that the correct peptide was synthesized.

Typical embodiments of the invention have been disclosed in the drawings and specifications. The use of specific terms is employed in a descriptive sense only, and these terms are not meant to limit the scope of the invention being set forth in the following claims.

1. A process for the solid-phase synthesis of peptides, comprising the step of:
applying infrared energy to a reaction vessel during said solid-phase synthesis of peptides.

2. The process of claim 1, wherein said step of applying infrared energy to a reaction vessel comprises heating one or more of a deprotection reaction, an activation reaction, a cleavage reaction, or a coupling reaction.

3. The process according to claim 1, wherein the contents of the reaction vessel are agitated during application of the infrared energy.

4. The process according to claim 1, wherein temperature in said reaction vessel is monitored in real time and the output of the infrared energy is adjusted to a predetermined point.

5. The process according to claim 2, wherein the deprotection reaction is monitored in real time and the duration or repetitions of one or more steps of deprotection or coupling is adjusted to a predetermined point.

6. A process for the solid phase synthesis of peptides, comprising:
performing a deprotection step while irradiating a reaction vessel containing an amino acid and deprotection reagents with infrared energy;
activating and coupling a second amino acid to a deprotected amine of said amino acid while irradiating said reaction vessel with infrared energy;
agitating said reaction vessel; and
performing successive deprotection and coupling steps while irradiating said reaction vessel until a desired peptide is synthesized.

7. The peptide synthesis process according to claim 6, wherein said agitating of the reaction vessel includes vortex mixing.

8. The peptide synthesis process according to claim 6, wherein said desired peptide is cleaved from the solid phase resin by mixing the resin with a cleavage solution while irradiating with infrared energy.

9. A peptide synthesizer reaction-heating platform, comprising:
one or more reaction vessels and a source of infrared energy configured to heat the contents of said one or more reaction vessels.

10. The platform of claim 9, wherein said source of infrared energy is disposed upon a hinged cover assembly such that the source is in near proximity to one or more reaction vessels when said cover assembly is in a closed position.

11. An instrument for solid phase peptide synthesis, comprising:
one or more reaction vessels;
a source of infrared energy configured to heat the contents of one or more reaction vessels;
and means for actuating and controlling fluid transfers and heating necessary for peptide synthesis.

12. The instrument of claim 11, further including a real-time infrared temperature sensor and
a means of agitating the content of said one or more reaction vessels.

13. The instrument of claim 12, further including control means for adjusting one or more of a reaction time and a temperature in response to said sensor.
